(19) United States
(12) Patent Application Publication
(10) Pub. No.: US 2017/0087182 A1
(43) Pub. Date: Mar. 30, 2017

(54) USE OF AN ANTIMICROBIAL COMPOSITION

(71) Applicant: AGXX Intellectual Property Holding GmbH, Berlin (DE)

(72) Inventors: Uwe Landau, Berlin (DE); Michael Lauenroth, Berlin (DE)

(21) Appl. No.: 15/314,129

(22) PCT Filed: May 28, 2015
(86) PCT No.: PCT/EP2015/061797

§ 371 Date: Nov. 28, 2016

(30) Foreign Application Priority Data
May 28, 2014 (EP) 14170308.2

(51) Int. Cl. A61K 33/38; A61L 15/44; A61K 9/00; A61K 33/24; A61K 31/375

(52) U.S. Cl. CPC A61K 33/38; A61K 33/24; A61K 31/375; A61K 9/0014; A61L 15/44; A61L 2300/404; A61L 2300/102; A61L 2300/104; A61L 2300/428

(57) ABSTRACT

The invention concerns the use of an antimicrobial composition comprising metallic silver and metallic ruthenium as well as at least one vitamin or at least one vitamin derivative for the topical treatment or prevention of skin, skin adnexa or mucosa diseases which are caused by infection with at least one microorganism. The invention also concerns a bandaging material or patch comprising an antimicrobial composition which comprises metallic silver and metallic ruthenium as well as at least one vitamin or at least one vitamin derivative. The antimicrobial composition (“AgXX”) has a broad-spectrum effect against bacteria (a: E. coli) and fungi (b: Pathogen is yeast and c: Penicillium).

a) Escherichia coli (additionally silver (“Ag”) as control),

b) Candida parapsilosis,

c) Penicillium notatum;

### BACKGROUND OF THE INVENTION

The invention concerns the use of an antimicrobial composition for the topical treatment or prevention of skin, skin adnexa or mucosa diseases which are caused by infection with at least one microorganism. The invention also concerns a bandaging material or patch comprising an antimicrobial composition.

### STATE OF THE ART

From WO 2008 046513 A2 a bioactive coating is known containing is silver, ruthenium and a vitamin used for the sterilization, disinfection and decontamination of water or aqueous solutions. The combination of silver with ruthenium and a vitamin or its derivative leads to the quicker and more efficient killing of microorganisms. At the same time, these bioactive metal surfaces prevent the infestation with microorganisms and the attachment or stable deposition of problematic biomolecules such as DNA, RNA or proteins. The coating produces a self-cleaning surface which upon contact with water or aqueous solutions very quickly and efficiently establishes and maintains its sterility over a long period of time.

Resistance to antibiotics in many pathogenic bacteria is a serious problem worldwide in the treatment of patients, and at present, there are often no satisfactory solutions. In particular, the greatest problems occur in case of urological infections and infections of the skin (WHO Library Cataloguing-in-Publication Data (2014): Antimicrobial resistance: global report on surveillance, World Health Organization, ISBN 978 92 4 156474 8). For that reason, there is an ever-increasing demand for therapies used in treating bacterial infections without the need of antibiotics.

But also in the case of viral infections, there is often a lack of alternatives to known therapies. One example are herpes infections. More than 85% of all Germans are infected by the Herpes simplex 1 virus (HSV 1); in about 12 million affected persons (about 20%), the illness breaks out regularly. In other countries, the figures are probably similar to those in Germany. Herpes viruses are regarded as the most common viruses worldwide. In Germany, cold sores (Herpes labialis) are among the most common forms of skin disease. is The primary infection is usually unnoticed in the form of a droplet or smear infection before the age of 6. After primary infection, the virus remains in the person's body for life. The outbreak period of Herpes is about 10-14 days and goes through 5 different stages: itching stage, blisters/progression stage, moisture/rupture stage, scabbing stage, and healing/resolution stage.

At present, treatment of the Herpes simplex 1 virus worldwide consists of two different strategies: Virostatica (ointments, tablets, suspensions) and Herpes patches with constituents to reduce symptoms during the course of the illness. Active substances include

Aciclovir and Penciclovir. Both substances are available in the form of creams and tablets; they inhibit the cell's metabolism, whereby the active substance is activated only in the infected cells. Herpes patches work on the basis of hydrocolloid therapy; they are applied to the affected area. They must adhere well and should be as invisible as possible. The patch includes substances which alleviate the course of the illness and reduce symptoms such as itching, swelling and reddening.

However, the treatment methods and active substances used thus far cannot significantly influence the course of the illness but only alleviate the unpleasant symptoms of labial herpes. That is why the search for a therapy approach continues that would diminish the outbreak of a cold sore immediately after its first onset (tingling, stretched skin or swelling) and prevents from the start the formation of blisters in the subsequent stage.

### DESCRIPTION OF THE INVENTION

The object of the invention is to provide alternative active substances and/or materials for the topical treatment or avoidance of skin, skin adnexa or mucosa diseases which are caused by infection with at least one microorganism.

According to the invention, this object is achieved by the appropriate therapeutic and preventive used of an antimicrobial composition comprising metallic silver and metallic ruthenium as well as at least one vitamin or at least one vitamin derivative. Surprisingly it has been found that a combination of the precious metals, silver and ruthenium, when activated by means of a vitamin, has a germicidal effect on all tested microorganisms and is excellently suited for the topical treatment of skin, skin adnexa or mucosa infections. This applies to humans as well as to animals. The topical application of the antimicrobial composition means that on the one hand, illnesses caused by microorganisms can be cured quickly and gently, and on the other hand, skin areas at risk, such as open wounds, can be very effectively treated to prevent infections. The composition comprising metallic silver and metallic ruthenium and at least one vitamin or at lest one vitamin derivate acts specifically against microorganisms and is harmless for the treated skin or mucous cells and for the skin adnexa.

For example, the microorganism can be a virus, a bacterium or a fungus. The effectiveness of the antibacterial composition against these types of microorganisms and in some cases also against their spore states could be confirmed in numerous tests. As shown in an investigation of the antibacterial composition and of silver by 13 is microbiologists from Germany and Spain, using 29 different bacteria, the antibacterial composition, for example in contrast to silver, has the effect of being very effective against antibiotics-resistant bacteria, and it very effectively prevents the formation of biofilm. As explained in greater detail below, the antibacterial composition is also effective against fungi and even against algae.

For example, the microorganism can also be the Herpes simplex 1 virus (Humanes herpes virus 1=HSV-1) which is known primarily as the cause of cold sores (Herpes labialis). But also about 20-30% of genital herpes infections (genital herpes=Herpes genitalis) are caused by HSV 1. Without treatment of a cold sore outbreak, the period until resolution is 10-14 days. Studies have shown that conventional herpes cremes do not or only slightly change the process of healing (by 0.5-2.0 days). However, for the patient, herpes blisters on the lips or in the face constitute physical, psychological and social stress. Patients are therefore giving top priority to shortening the course of the illness. It has been demonstrated that an antibacterial composition comprising metallic silver and metallic ruthenium and at least one vitamin or at least one vitamin derivative is so effective against the Herpes simplex 1 virus that it completely contains the herpes within one day after the first symptoms appear. Contrary to the herpes treatment solutions already available in the market, the antimicrobial composition can shorten the herpes illness by several days (8.5-12 days).

But for example, the microorganism can also be the Herpes simplex 2 virus (Humanes herpes 2 virus=HSV 2) which is responsible for the vast majority of genital herpes manifestations (penis, vagina and anal is region). The antibacterial composition comprising metallic silver and metallic ruthenium and at least one vitamin or at least one vitamin derivative is also very effective against the Herpes simplex 2 virus and is therefore also an effective agent against genital herpes (Herpes genitalis).

In an advantageous embodiment of the invention, it is provided that the antimicrobial composition is applied to at least one surface of a carrier material. For example, the carrier material can be a tissue, a foil, a mesh, a bandaging material or a patch (plaster, band-aid®). The carrier material can also, at least partially, consist of metal, plastic, glass, ceramic, natural fibres or other suitable materials.

For example, the carrier material can be such that it is wettable and/or permeable for moisture. Moisture promotes or increases the effect of the antimicrobial composition, and it is therefore an advantage if the carrier material can absorb moisture and can practically serve as a moisture reserve. In addition or alternatively, the carrier material can also be such that it is moisture-permeable, allowing the antimicrobial composition and/or the skin or mucous surface to be wettable from the outside even though it is covered with the carrier material.

In an advantageous embodiment of the invention, the carrier material can also comprise a material for retaining moisture such as polymers that contain water but are water-insoluble (so-called hydrogels).

Another advantageous embodiment of the invention provides that the carrier material is gas- and air-permeable since oxygen, for instance, is of special advantage for the effectiveness of the antibacterial composition.

Another advantageous embodiment of the invention provides that the antimicrobial composition comprises at least one silver layer with at least one ruthenium layer applied to it whereby the silver layer remains uncovered by the ruthenium layer in at least one partial sector. This allows the ruthenium layer to be applied to the silver layer, for instance, in the form of islands or pods, so to speak in clusters. This can warrant an optimal effectiveness of the antibacterial composition. For example, the antibacterial composition can be created by applying a ruthenium layer to a silver-containing surface or to a silver-containing carrier material, or by applying a silver coating to the carrier material, and by subsequently applying a ruthenium layer upon the silver coating, or by applying ruthenium/silver particles to a surface or to the carrier material. The thus produced Ag/Ru surface is then activated by applying at least one vitamin or at least one vitamin derivate. The layers, i.e. the ruthenium layer and a silver under-layer that may be required (usually 2-10 μm in thickness), are preferably applied by galvanic deposition or are applied by electroplating. Other coating s methods such as PVD, CVD, sputter, sol-gel and reduction process methods, are also plating processes suitable for the purpose.

In another advantageous embodiment of the invention, it is provided that the antimicrobial composition comprises at least one silver wire or at least a silver-coated wire covered partially with a ruthenium layer. Instead of a silver wire, a silver-coated high-grade steel wire can be used, for instance, which is then partially coated with ruthenium. This Ag/Ru wire is then activated by applying at least one vitamin or at least one vitamin derivative. Then the wire-shaped antimicrobial composition can, for instance, be processed and used as a component of a mesh or sieve.

The surface to be coated with ruthenium, which may, for instance, comprise a silver-containing surface or silver surface, can be any material (such as metal or plastic) with a thin silver-containing coating applied, which, together with the applied outer ruthenium layer forms an effective ruthenium/silver sandwich system, with a silver or silver alloy sub-layer or base and a moisture- or wetness-permeating ruthenium outer layer, top layer or covering layer.

In another advantageous embodiment of the invention it is provided that the antimicrobial composition comprises silver/ruthenium bimetal particles that have been re-treated with the vitamin or with the vitamin derivative. Such particles can, for instance, be used as component of a powder, a suspension, a cream, an ointment or a gel.

In another advantageous embodiment of the invention it is provided that the antimicrobial composition, in whatever form, is contained in a suspension, a cream, an ointment, a gel or a powder and is used for the topical treatment of microbial illnesses of the skin, skin adnexa or mucosa in humans or animals.

In another advantageous embodiment of the invention it is provided that the vitamin is at least one compound from the group of water-soluble vitamins or a salt or acid derivative of that compound. In this embodiment of the invention, other than with the known oligodynamic effect of silver, the antimicrobial effect of the composition, due to a completely different active mechanism, is considerably improved, for is instance by the combination of silver and ruthenium and a vitamin such as ascorbic acid or its derivatives. The antibacterial composition leads to the quicker and more efficient killing of microorganisms. At the same time such antibacterial compositions inhibit the infection of sensitive or weakened skin and mucous surfaces with microorganisms and the attachment or stable deposition of biomolecules such as DNA, RNA or proteins on the skin or on mucosa.

For instance, a silver/ruthenium surface can be treated with at least one water-soluble vitamin, or it comprises that or additional substances, such that the surface is activated by such substances, causing an unexpectedly strong antimicrobial effect of the composition. Surprisingly it has also been found that only a non-degraded water-soluble vitamin causes the unexpected antimicrobial effect in conjunction with silver and ruthenium. Many measurements have shown that the re-treatment of a ruthenium-coated silver surface, for instance with ascorbic acid, significantly changes the surface and causes the antimicrobial effect to work according to a completely different mechanism than with classic silver technology (classic oligodynamic silver technology=release of silver ions). There are indications that the effect of the antimicrobial composition originates in a micro-electric field and that the wetting behaviour of the surface, for instance, is distinctly changed, when we compare a purely silver/ruthenium surface with the activated antimicrobial composition. In measuring with the EM atomic force microscope, significant differences have also been found which give a clear indication that a micro-electric field is established through the combination of silver/ruthenium with a water-soluble vitamin, and this has not been observed with pure silver/ruthenium surfaces. There is a cathodic oxygen reduction which leads to the formation of free oxygen radicals (English: reactive oxygen species=ROS) such as H₂O₂. At the same time, the antibacterial composition leads to the formation of a chemically very stable surface that is reaction-resistant against sulphides and chloride. The high stability paired with a long-lasting strong antimicrobial effectiveness has been confirmed in many years of antimicrobial uses of the antibacterial composition in diverse aqueous solutions (such as cooling lubricant solutions, process water with corrosion protection agents and emulsifiers of the most various kinds, or urine).

In another advantageous embodiment of the invention it is provided that the vitamin is ascorbic acid or an ascorbic acid derivative.

According to the invention, the object is also achieved by bandaging material or a patch (plaster, band-aid®) containing an antimicrobial composition comprising metallic silver and metallic ruthenium as well as at least one vitamin or at least one vitamin derivative. Such a bandage or patch is suited advantageously for the application and topical use of the antimicrobial composition for the effective treatment of illnesses of the skin, skin adnexa or mucosa and also to protect skin areas such as open wounds, which are at risk of certain infections.

In the sense of the invention, “topical treatment” means the application of active mechanisms where they are to have a therapeutic effect, in contrast to a so-called systemic treatment, i.e. using medication such as infusion or tablets. Thus, topical treatment is a local form of therapy used in particular in dermatology, ophthalmology, otolaryngology and gynaecology, i.e. for body regions accessible from the outside such as the skin, the cornea or mucosa. Forms of topical treatment have the is advantage that the active substances or mechanisms are effective only where they are needed while sparing the healthy skin or mucosa. Thanks to this local application, many systemic side effects can be prevented as well.

The term “skin” as used in the sense of the invention, comprises the external organ of a human or animal body which among other functions serves to delineate the outside of the body from the inside. The term “skin” includes all layers of the organ, especially the epidermis (Stratum corneum), the dermis and the subcutaneous tissue. In the sense of the invention, the term “skin” in particular also includes the surface of the lips and labia and of the outer and middle ear.

The term “skin adnexa” in the sense of the invention includes the hair with the sebaceous glands and the hair arrector muscles (Musculus arrector pili), nails, horns and sweat glands (including mammary glands) of the human or animal body.

The term “mucosa” in the sense of the invention comprises the protective layers (Tunica mucosa) which line the interior of the hollow organs of the human or animal body and which produce or secrete mucus, in particular also the conjunctiva, the oral mucosa, the foreskin and the vaginal mucosa.

Below, the invention is described in detail, by example and with reference to the Figures.

### SHORT DESCRIPTION OF THE FIGURES

In the figures:

FIG. 1 shows a photographic view of a self-adhesive foil coated with the antimicrobial composition on a hydrogel background (moisture retention);

FIG. 2 shows a photographic view of an applied carrier material with antimicrobial composition;

FIG. 3 shows photographic views of the healing process of a labial herpes being treated with the antimicrobial composition;

FIG. 4 shows photographic views of cultures with microorganism colonies around carrier materials coated with the antimicrobial composition (“AgXX” =activated Ag/Ru composition),

a) Escherichia coli (additionally silver (Ag) as a control),

b) Candida parapsilosis,

c) Penicillium notatum;

FIG. 5 shows microscopic views of yeast cells after 15 minutes exposure to a powdery antibacterial composition and to nanosilver (dark areas),

a) activated Ag/Ru composition,

b) nanosilver (control);

FIG. 6 shows an enlarged section (rectangular box) with killed yeast cells (dark grey) and a cell prior to dying (light cell), and

FIG. 7 shows microscopic views of algae cells around a bead coated with the antimicrobial composition (dark circle)

(a) at the beginning of the experiment,

(b) after 3 minutes.

### DESCRIPTION OF EXAMPLES AND PREFERRED EMBODIMENTS OF THE INVENTION

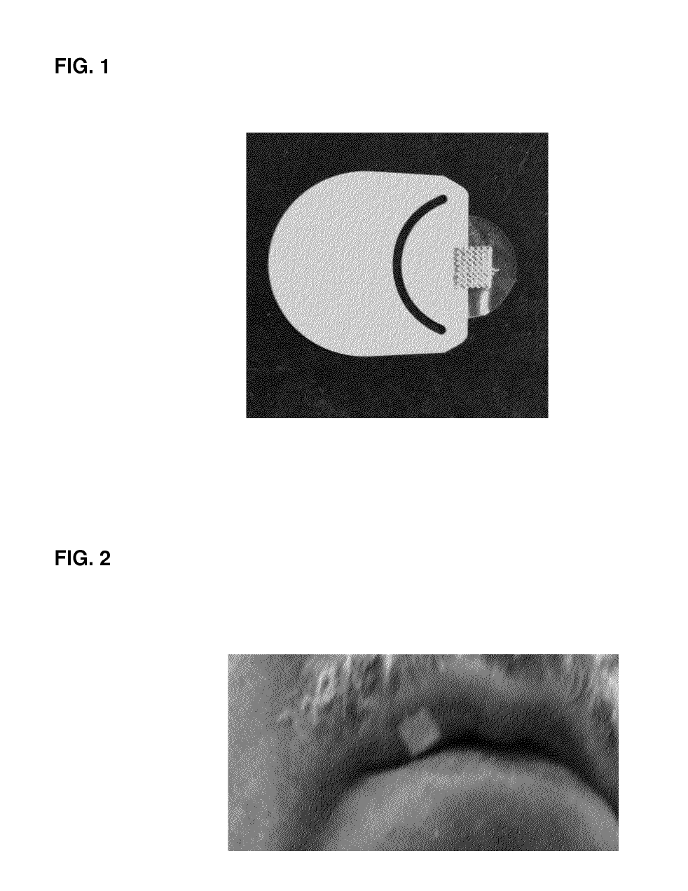

FIG. 1 shows a photographic view of a self-adhesive foil coated with the antimicrobial composition on a hydrogel background. In this embodiment, the carrier material for the activated antimicrobial Ag/Ru composition is a self-adhesive foil by which the composition can be easily applied to the surface such as the skin to be treated. In this or similar fashion, the antibacterial composition can be applied in the form of a first-aid bandage or patch (plaster, band-aid®). The hydrogel background serves to retain moisture which promotes or increases the effect of the antimicrobial composition.

FIG. 2 shows a photographic view of an applied carrier material with antimicrobial composition. Here, the carrier material was applied to the skin of the lips of a human patient, for the topical treatment of labial herpes.

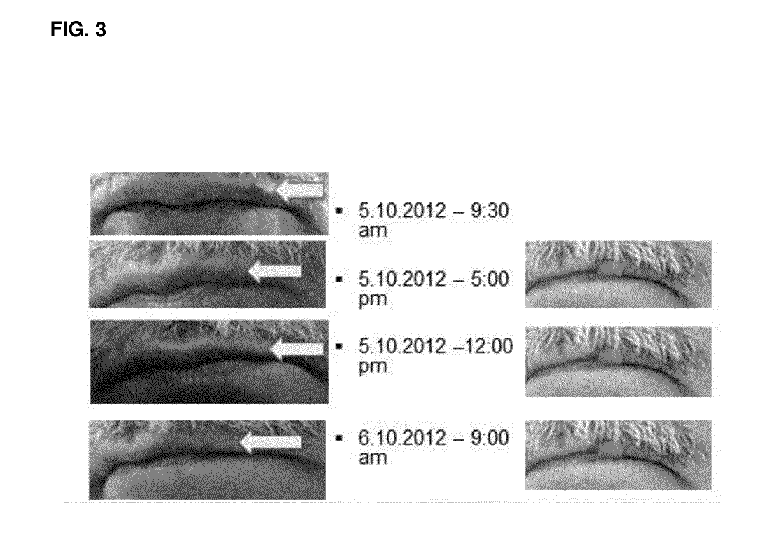

FIG. 3 shows photographic views of the healing process of a labial herpes being treated with the antimicrobial composition. After the first symptoms of a beginning labial herpes, a carrier material with activated antimicrobial Ag/Ru composition was applied to the skin of the lips of a human patient (right views), and the course of the illness is documented over a period of a few hours (left views). It was found that the topical application to the skin of the antimicrobial composition according to the invention already caused the symptoms to subside completely within less than 24 hours. Using the herpes patch according to the invention distinctly shortened the course of the illness, with stages 3 to 5 of the herpes outbreak not occurring at all if the patch is applied on time. It is a clear advantage in comparison with previous herpes products that the carrier material can be re-used during herpes treatment. To the patients, both factors mean a distinct advantage in terms of quality living and cost savings.

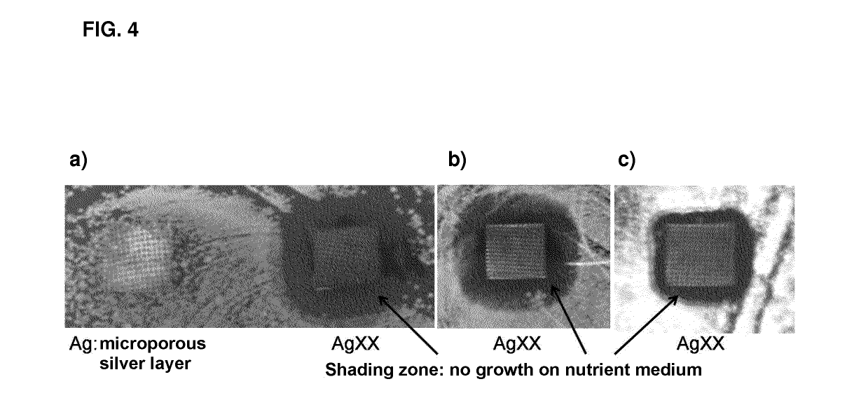

FIG. 4 shows photographic views of cultures with microorganism colonies around carrier materials coated with the antimicrobial composition. It was found that the antimicrobial composition has a broad-band effect against bacteria (a: E. coli) and fungi (b: pathogenic yeast and c: Penicillium. Here we used the antimicrobial composition in the form of coated wire mesh laid upon the cultures infested with the microorganisms in question. After an incubation period of 36 hours (106/ml, 30° C.), distinctive bacteria-free areola showed all around the wire mesh sections according to the invention, which proves the antimicrobial effect of the coated wire mesh. By contrast, it was found that with a wire mesh provided with a microporous silver coating (a: left-control) the microorganisms could not be killed, and it was being completely covered by the microorganisms.

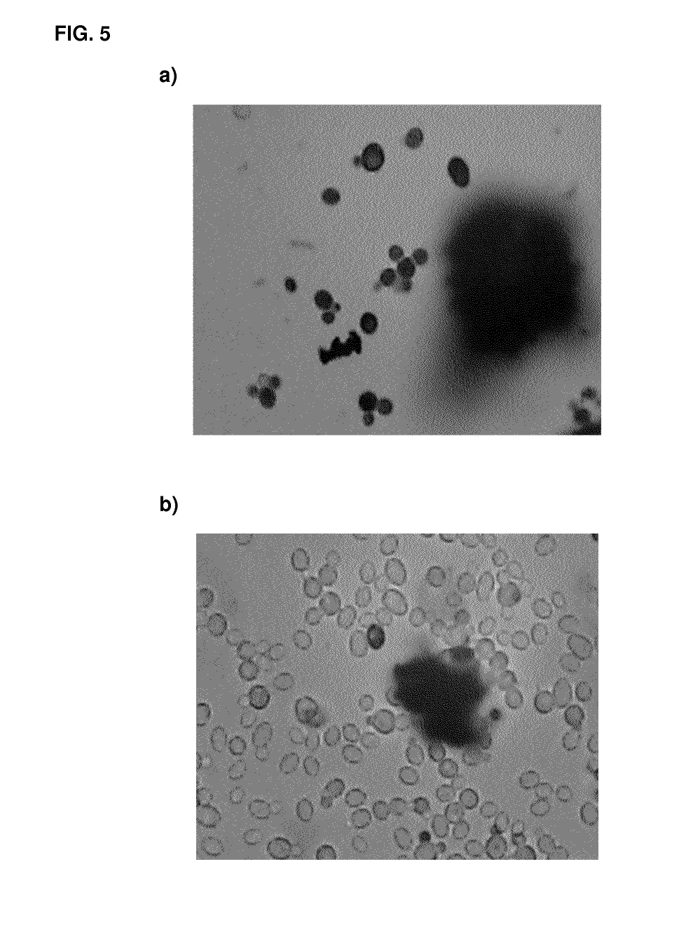

FIG. 5 shows killed yeast cells after 15 minutes exposure to a powdery antibacterial composition (dark area). The aqueous solution, in which the yeast cells had been, contained a dye (methylene blue) which could only have penetrated the killed cells through the cell wall. Due to the changed pH in the cell, the dye reacts and forms its characteristic blue colour. In FIG. 5a, yeast cells were brought in contact with the activated Ag/Ru powder. After 15 minutes, all the yeast cells within about 50 μm around the powdery antimicrobial composition had been dyed by the methylene blue dye. This dye effect shows the yeast cells were killed, for the dye can only penetrate the cell when the cells are dead and their cell wall is damaged. Since the dye process takes a while, the yeast cells were killed within less than 15 minutes. In contrast, with the effect of nanosilver (FIG. 5b: control) no microorganisms had been killed even after 60 minutes. The individual dead yeast cells were killed already when the experiment started and only show that in this aqueous solution, the dye for the killed microorganisms had also been present.

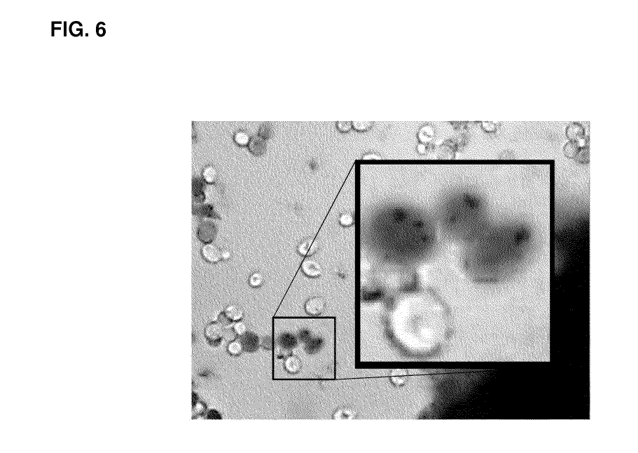

FIG. 6 shows an enlarged section (rectangular box) with killed yeast cells (dark grey) and a cell prior to dying (light cell). The dark dots in the cell walls can be interpreted as pores. Since the blue coloration takes some time after the cells are killed, the light cell seen in the enlarged section is possibly already dead but not yet coloured, as the first dark dots in the membrane indicate.

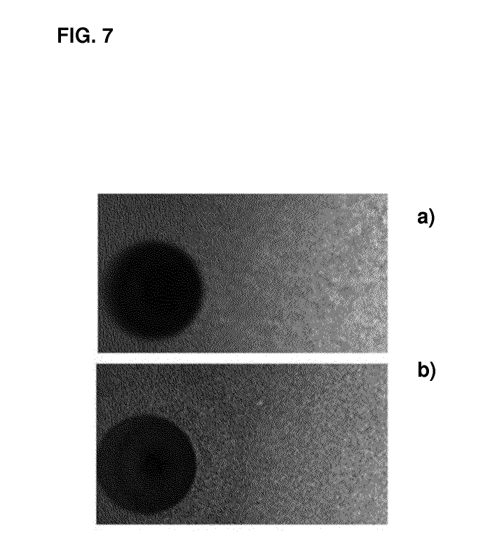

FIG. 7 shows microscopic views of algae cells around a bead coated with the antimicrobial composition (dark circle). At the beginning of the experiment (FIG. 7a) the algae are intact while already 3 minutes later, dead algae cells are shown all around the bead (FIG. 7b). This shows therefore that using an activated Ag/Ru composition according to the invention also kills algae cells.

In other experiments not illustrated here, we could also show that the is use of an activated Ag/Ru composition according to the invention also has an antimicrobial effect on Legionella.

All the experiments conducted have made it clear that the antimicrobial composition obviously leads to the destruction of the cell wall and/or the cell membrane of the microorganisms in question, causing irreparable damage of the cells, effectively killing them.

In spite of this strong antimicrobial effect, the inventive use of the activated Ag/Ru composition is harmless for the humans or animals treated with it. In a study on cell toxicity with antimicrobially coated glass beads, experiments were conducted with MRC-5 cell lines. The experiments were conducted according to ISO 10993, ISO 10993-12 and USP Chapters 87 and 1031. The examination showed a level 1 cytotoxic effect (slight cytotoxicity), which is regarded as harmless, thus allowing an application in human patients.

The first in vivo experiments with rats have confirmed the compatibility of the antimicrobial composition. The experiments were conducted with activated Ag/Ru glass beads in the bladders of rats. There were no cytotoxically negative findings that would contradict the use of the antimicrobial composition for urological purposes.

### EXAMPLE

Use of the antimicrobial composition as a herpes patch:

Herpes simplex 1 is a self-limited illness. Antiviral therapy is not absolutely necessary. Many patients suffering from recurrences are is not consulting a physician, but take over-the-counter preparations.

With an almost 90% infection rate in the European population with Herpes simplex viruses and an annual incidence of up to 30% of the affected population, the question is raised again and again for the most effective therapy. Especially the Herpes simplex 1 virus (HSV 1) is widespread and stressful for those affected due to the typical infection of epithelial cells in the skin and associated neurons in the mouth region. An American study has shown an endogenous re-infection in about 30% of infected patients, with the recurring infection rate near 1%. For the most part, the infection occurs on and around the lips. Due to its wide distribution, the Herpes simplex disorder is not only a health problem for the affected person, but also a social stress factor. The herpes products in the market so far only achieve a short reduction in the course of the illness and can at best alleviate the symptoms of herpes lip blistering.

The advantageous possibility of applying an activated Ag/Ru composition upon a large variety of carrier materials allows the concept of a completely new type of herpes patch. The basic carrier is a microporous polymer foil customary in medical technology and commercially available, which is provided with a skin-compatible adhesive on one side. On it, a thin film of a hydrogel is applied which gives sufficient adhesion to a carrier material (such as tissue) coated with activated silver/ruthenium that serves as a moisture reserve. Moisture increases the effectiveness of the antimicrobial composition. The Ag/Ru tissue can also be easily removed from the gel to apply it to another carrier foil as soon as the herpes patch's adhesive strength is gone from the area to be treated.

The novel Ag/Ru patch is applied to the affected area in the face in the same way as with classic patches. Since the effectiveness of the antimicrobial composition is supported by moisture, the moisture can also be applied from the outside by wetting the moisture-permeable patch with water and thus bringing moisture to the activated Ag/Ru foil and the skin.

A disadvantage of the Penciclovir substance is that it must be applied every 2 hours. The best known herpes patches have adhesion power for a maximum of 8 hours at the herpes site, after which the herpes patch mist be replaced. With the Ag/Ru patch according to the invention, the patch has about the same effective period on the herpes site. However, the active medium (Ag/Ru tissue) is only transferred from one adhesive carrier to the next without having to use a new Ag/Ru tissue. This means that during the entire healing period, only one Ag/Ru active medium can be enough. Only the adhesive carrier is renewed, which represents a considerable cost advantage. Furthermore, the application is very easy to accomplish.

In various experiments conducted by ourselves, the high effectiveness of the Ag/Ru herpes patch could be confirmed many times (see FIG. 3).

Advantages of the Ag/Ru patch and its use according to the invention:
Short application period until successful repression of the herpes,
Simple handling,
No side effects to be expected,
Completely new active mechanism in comparison with conventional anti-herpes systems,
This Ag/Ru herpes patch is based on an especially designed coating system of precious metals and a vitamin derivative. This structured material combination provides for the activated Ag/Ru system a very high degree of effectiveness against microorganisms. An important part is assumed by the formation of a micro-electric field on the Ag/Ru surface which strengthens the catalytic effect of the Ag/Ru surface.
The Ag/Ru surface for the Herpes patches is largely produced automatically in a roll to roll coating process.

Alternatively to a use of the antimicrobial composition in the form of a band-aid® (plaster) type of patch, the following forms of application are possible as well:

Ointment (including fatty ointments), cream, lotion/milk, liquid (including solution and/or suspension; on water, alcohol or aromatic base), shaking mixture (lotion), solid (such as powder), paste, taps, eye drops or eye ointment, or as local therapy for ears, nose and throat in the form of drops or spray.

1. Method for treating or avoiding skin, skin adnexa or mucosa diseases comprising:
applying an antimicrobial composition comprising metallic silver and metallic ruthenium as well as at least one vitamin or at least one derivative of a vitamin topically to treat or avoid skin disease, skin adnexa or mucosa diseases which are caused by infection with at least one microorganism.

2. The method according to claim 1, wherein the microorganism is a virus, a bacterium or a fungus.

3. The method according to claim 1, wherein the microorganism is a herpes simplex 1 virus.

4. The method according to claim 1, wherein the antimicrobial composition is applied onto at least one surface of a carrier material.

5. The method according to claim 4, wherein the carrier material is a tissue, a foil, a mesh, a bandaging material, a compress, or a patch.

6. The method according to claim 4, wherein the carrier material is wettable and/or permeable to moisture.

7. The method according to claim 4, wherein the carrier material comprises a material for storing moisture.

8. The method according to claim 4, wherein the carrier material is permeable to gas and/or air.

9. The method according to claim 1, wherein the antimicrobial composition comprises at least one silver layer having a ruthenium layer applied thereon, wherein the silver layer is not covered by the ruthenium layer in at least one subarea.

10. The method according to claim 1, wherein the antimicrobial composition comprises at least one silver wire or at least a silver-coated wire, which is partially covered by a ruthenium layer.

11. The method according to claim 1, wherein the antimicrobial composition comprises silver-ruthenium bimetallic particles.

12. The method according to claim 1, wherein the antimicrobial composition is included in a suspension, a cream, an ointment, a gel, or a powder.

13. The method according to claim 1 wherein the vitamin is at least one compound selected from the group consisting of water-soluble vitamins, and a salt or acidic derivatives thereof.

14. The method according to claim 12, wherein the vitamin is ascorbic acid or a derivative of ascorbic acid.

15. Bandaging material or patch, comprising an antimicrobial composition comprising metallic silver and metallic ruthenium as well as at least one vitamin or at least one derivative of a vitamin.

16. The bandaging material or patch according to claim 15, wherein the bandaging material or patch is permeable to gas and/or air.

17. The bandaging material or patch according to claim 15, wherein the antimicrobial composition comprises at least one silver layer having a ruthenium layer applied thereon, wherein the silver layer is not covered by the ruthenium layer in at least one subarea.

18. The bandaging material or patch according to claim 15, wherein the antimicrobial composition comprises at least one silver wire or at least a silver-coated wire, which is partially covered by a ruthenium layer.

19. The bandaging material or patch according to claim 15, wherein the antimicrobial composition comprises silver-ruthenium bimetallic particles.
